(19) United States
(12) Patent Application Publication
(10) Pub. No.: US 2026/0034995 A1
(43) Pub. Date: Feb. 5, 2026

(54) VEHICLE CONTROLLER

(71) Applicant: TOYOTA JIDOSHA KABUSHIKI KAISHA, Toyota-shi (JP)

(72) Inventor: Mina MIZUNO, Nagoya (JP)

(73) Assignee: TOYOTA JIDOSHA KABUSHIKI KAISHA, Toyota-shi (JP)

(21) Appl. No.: 19/225,125

(22) Filed: Jun. 2, 2025

(30) Foreign Application Priority Data
Aug. 1, 2024 (JP) 2024-126053

(51) Int. Cl. B60W 40/08; A61B 5/00; B60Q 5/00; B60W 50/14

(52) U.S. Cl. CPC B60W 40/08; A61B 5/4809; A61B 5/741; B60Q 5/005; B60W 50/14; A61B 2503/22; B60W 2040/0818; B60W 2050/143; B60W 2540/229; B60W 2710/30

(57) ABSTRACT

A vehicle controller includes a processor, which acquires biological data of a user of a vehicle from a sensor provided in the vehicle; determines a consciousness level of the user based on the biological by conducting a kinesiology test; and controls an interface mounted on the vehicle in accordance with the determined consciousness level to change an in-vehicle environment.

### CROSS-REFERENCE TO RELATED APPLICATION(S)

The present application claims priority to and incorporates by reference the entire contents of Japanese Patent Application No. 2024-126053 filed in Japan on Aug. 1, 2024.

### BACKGROUND

The present disclosure relates to a vehicle controller.

Japanese Laid-open Patent Publication No. 2008-126818 discloses a technique for obtaining biological information of a user and autonomously controlling an operation of an in-vehicle device based on the biological information.

### BACKGROUND

There is a need for providing a vehicle controller that can provide an in-vehicle environment according to a user's desire.

According to an embodiment, A vehicle controller includes a processor, which acquires biological data of a user of a vehicle from a sensor provided in the vehicle; determines a consciousness level of the user based on the biological by conducting a kinesiology test; and controls an interface mounted on the vehicle in accordance with the determined consciousness level to change an in-vehicle environment.

### BRIEF DESCRIPTION OF THE DRAWINGS

FIG. 1 is a block diagram illustrating a schematic configuration of a vehicle controller according to an embodiment;

FIG. 2 is a flowchart illustrating a flow of processing of a vehicle control method that is executed by the vehicle controller according to the embodiment;

FIG. 3 is a flowchart illustrating a flow of processing of a first kinesiology test in the vehicle control method that is executed by the vehicle controller according to the embodiment;

FIG. 4 is a flowchart illustrating a flow of processing of a second kinesiology test in the vehicle control method that is executed by the vehicle controller according to the embodiment; and

FIG. 5 is a flowchart illustrating a flow of processing of a third kinesiology test in the vehicle control method that is executed by the vehicle controller according to the embodiment.

### DETAILED DESCRIPTION

In the technique disclosed in Japanese Laid-open Patent Publication No. 2008-126818, although it is possible to grasp the apparent consciousness of the user through the biological information, since it is not possible to grasp the deeper latent consciousness than that, it is difficult to control the operation of the onboard device as desired by the user.

A vehicle controller according to an embodiment of the present disclosure will be described with reference to the drawings. In addition, components in the following embodiments include those which can be easily substituted by those skilled in the art or those which are substantially the same.

### Vehicle Controller

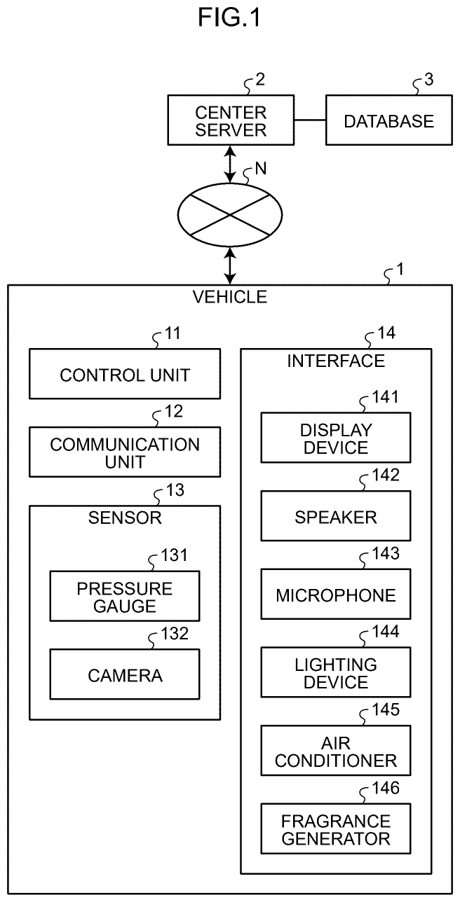

A configuration of the Vehicle controller according to the embodiment will be described with reference to FIG. 1. The Vehicle controller according to the embodiment determines the consciousness level of a user (for example, a driver, etc.) of a vehicle through a kinesiology test, and controls an interface in the vehicle according to the determined consciousness level.

The kinesiology (Physical Kinematics) test indicates that the muscle was tested for external stimuli (by David R. Hawkins, “Power vs. Force, Revised Edition (kindle)”, Natural Spirit Corporation, Aug. 18, 2019, see pp. 80-125). In the kinesiology test, for example, a positive stimulus on the body causes a strong muscle response, while a negative stimulus on the body causes a weak muscle response. In addition, the “consciousness level” in kinesiology test indicates the state of latent consciousness deeper than the user's apparent consciousness, not the level of the user's arousal state. In kinesiology tests, by focusing on the user's muscle response, it is possible to determine whether the external environment (in this embodiment, the in-vehicle environment) is appropriate for the user (optimal) or not, under the latent consciousness that the user does not perceive itself.

For example, even if the user is satisfied with the present in-vehicle environment (e.g., the type and the volume of Back Ground Music (BGM) in the vehicle, the brightness in the vehicle, the temperature in the vehicle, etc.) (they are satisfied with the obvious consciousness), the body of the user may be subject to accumulation of strain, fatigue, stress, etc. For example, in midsummer when the air temperature is high, the air conditioning in the car is often increased. However, if the air conditioner is used for a long time in the car with the air conditioning, the skin may dry and other physical conditions may deteriorate. This embodiment provides a truly beneficial in-vehicle environment for the user by determining, through kinesiology tests, the reaction of the body (muscles) that the user cannot easily perceive.

The vehicle controller according to the embodiment is applied to the vehicle 1, for example, a common engine vehicle (conveyer vehicle), a Hybrid Electric Vehicle (HEV), a Plug-in Hybrid Electric Vehicle (PHEV) or the like. The vehicle 1 may be, for example, a Fuel Cell Electric Vehicle (FCEV), a Battery Electric Vehicle (BEV), or the like.

A vehicle 1, for example, through a network N such as the Internet line network, a cellular phone line network, is configured to be able to communicate with each other with a center server 2. The center server 2 is connected to a database 3.

The vehicle 1 includes a control unit 11, a communication unit 12, a sensor 13, and an interface 14. In FIG. 1, among the actual configuration of the vehicle 1, it is to be noted that only the configurations necessary for realizing the vehicle controller according to the embodiment are illustrated, and other configurations are omitted.

The control unit 11 is an Electronic Control Unit (ECU) which includes a microcomputer having, for example, a Central Processing Unit (CPU), a Read Only Memory (ROM), a Random Access Memory (RAM) or the like as its main components.

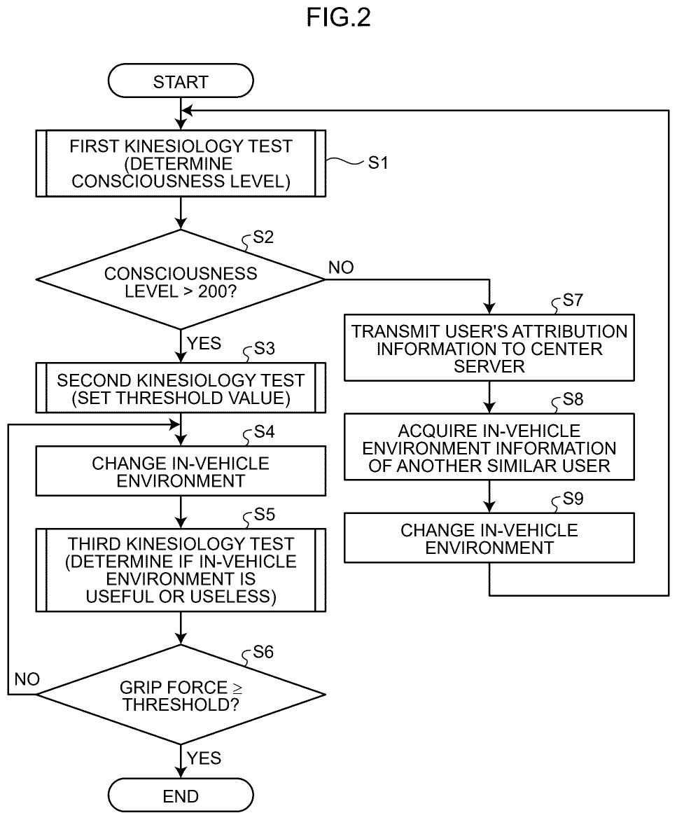

The control unit 11 performs the following processing. In the following description, if necessary, step numbers of the flowchart of FIG. 2 to FIG. 5 corresponding to each process are shown.
(1) Bio-data of the user of the vehicle 1 is acquired from the sensor 13 provided in the vehicle 1 (step S13);
(2) Based on kinesiology test, user's consciousness level is judged from acquired biomedical data (steps S14 to S19); and
(3) Depending on the determined consciousness level, the in-vehicle environment is changed by controlling the interface 14 mounted on the vehicle 1 (step S4).

Here, the processing of determining the above-described consciousness level includes the following processing.
(1) Outputting a predetermined sound during measuring the biological data by the sensor 13 (steps S15 and S16); and
(2) Determine the consciousness level based on the biometric data measured after outputting the predetermined audio (steps S17 to S19).

Further, the processing of determining the above-described consciousness level includes the following processing.
(1) During the measurement of the biometric data by the sensor 13, a first voice (sound) that satisfies a predetermined consciousness level and a second voice that does not satisfy a predetermined consciousness level are output (steps S15 and S16);
(2) The value of the first biological data measured after the output of the first sound and the value of the second biological data measured after the output of the second sound are compared (step S17);
(3) When the value of the first biological data is larger than the value of the second biological data (Yes in step S17), it is determined that the consciousness level is a predetermined consciousness level (step S18); and
(4) When the value of the first living body data is equal to or less than the value of the second living body data (No in step S17), the interface 14 is controlled to change the value to the previously applied in-vehicle environment for the user having the property similar to the user (step S9).

In addition, the processing to change the in-vehicle environment described above includes the following processing.
(1) By controlling the interface 14, the in-vehicle environment is changed (step S4).
(2) Measuring biological data after changing the in-vehicle environment (step S33).
(3) If the value of the measured biological data is equal to or greater than the predetermined threshold value, the in-vehicle environment after the change is continued, and if the value of the measured biological data is less than the predetermined threshold value, the change to another in-vehicle environment and the measurement of the biological data are repeated until the value of the biological data is equal to or greater than the predetermined threshold value (steps S4 to S6).

In addition, the processing to change the in-vehicle environment described above includes the following processing.
(1) If the changed in-vehicle environment is accepted by the user, the changed in-vehicle environment is continued.

The communication unit 12 is composed of, for example, a Data Communication Module (DCM) or the like. The communication unit 12 communicates information with the center server 2 through the network N.

The sensor 13 is for acquiring the biological information of the user. The sensor 13 includes a pressure gauge 131 and a camera 132. The pressure gauge 131, for example, is incorporated in the steering of the vehicle 1 and detects a grip strength (force) of the user when the user grips the steering. The camera 132 is installed at a position where the user's body can be imaged, for example, in a vehicle cabin. The camera 132 images the facial expression of the user, the size of the pupil, the movement of the body such as the arm, the color of the skin (blood color), and the like.

The interface 14 is, for example, a vehicle-mounted device provided in the vehicle interior of the vehicle 1. The interface 14 includes a display device 141, a speaker 142, a microphone 143, a lighting device 144, an air conditioner 145, and a fragrance generator 146.

The display device 141 is, for example, a liquid crystal display (LCD), an organic EL display (OLED) or the like. The speaker 142 utters a predetermined phrase to the user under the control of the control section 11, for example, during the kinesiology test. The microphone 143 records a user's utterance and transmits it to the control section 11, for example, during kinesiology testing. The lighting device 144, air conditioner 145, and the fragrance generator 146 are provided in the vehicle interior.

The center server 2 is realized by a general-purpose computer, such as a workstation or a personal computer. When attribute information of the user is received from the vehicle 1, the center server 2 acquires the in-vehicle environment information of another user similar to the user from the database 3 and transmits the in-vehicle environment information to the vehicle 1.

The “attribution information” includes, for example, physical features (e.g., age, gender, physical form, etc.), mental features (e.g., personality, behavior patterns, etc.) of a user, and the like. The “in-vehicle environmental information” is information about the in-vehicle environment of a plurality of users who ride on the vehicle 1 in the past. The in-vehicle environmental information includes information about, for example, the type and the volume of BGM in the vehicle, the brightness in the vehicle, the temperature in the vehicle, the air conditioner air volume in the vehicle, the fragrance in the vehicle, and the like. In the database 3, the in-vehicle environment information previously collected from a plurality of users is stored and classified by attribute information.

### Vehicle Control Method

A flow of a vehicle control method in which the vehicle controller according to the embodiment executes will be described with reference to FIG. 2. In the following description, an example of using the grip strength as the biological data of the user, that is, an example of performing kinesiology test using the g of the user acquired by the pressure gauge 131 will be described.

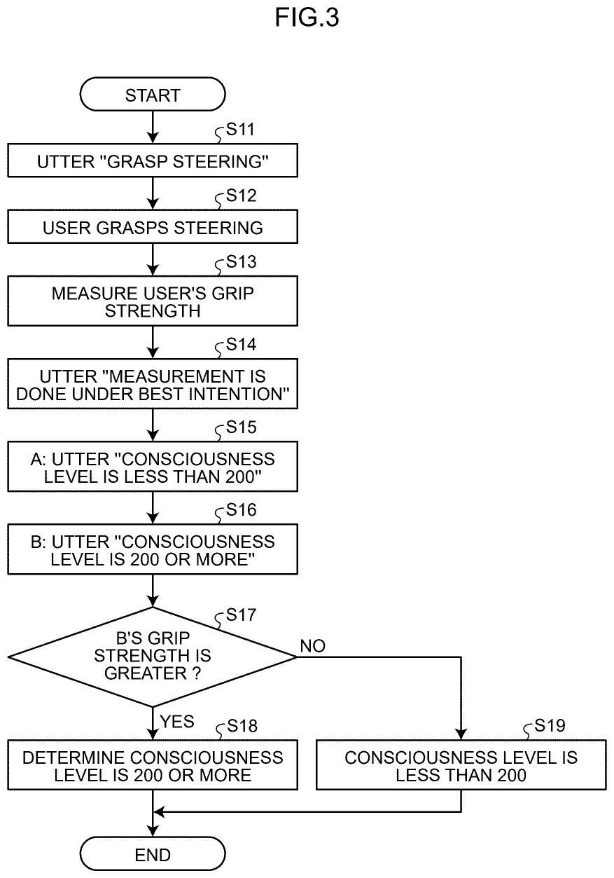

First, the control unit 11 performs a first kinesiology test (step S1). The first kinesiology test is a test for judging the user's current consciousness level. The first kinesiology test will be described in detail later (see FIG. 3). Subsequently, the control unit 11 determines whether the consciousness level determined by the first kinesiology test is equal to or more than 200 (step S2).

In the vehicle control method according to the embodiment, kinesiology tests are performed three times in total, as indicated in FIG. 2. Then, the determination in step S2 is required to proceed to the two kinesiology tests after step S3. That is, only when the consciousness level is determined to be 200 or more in step S2, other kinesiology tests are performed after step S3, and when the consciousness level is determined to be less than 200, the kinesiology tests are not performed thereafter.

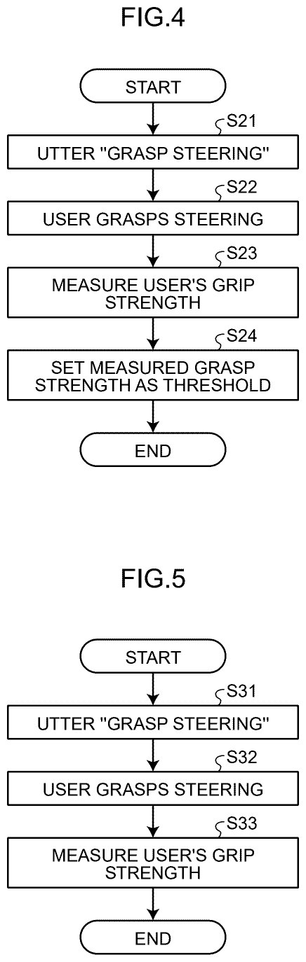

In step S2, when it is determined that the consciousness level is 200 or more (Yes in step S2), the control unit 11 performs the second kinesiology test (step S3). The second kinesiology test is a test to set the threshold. The second kinesiology test will be described in detail later (see FIG. 4). Subsequently, the control unit 11 controls the interface 14 to change the in-vehicle environment (step S4).

Subsequently, the control unit 11 performs a third kinesiology test (step S5). The third kinesiology test is a test to determine whether the in-vehicle environment changed in step S4 was useful or useless for the user. The third kinesiology test will be described in detail below (see FIG. 5).

Subsequently, the control unit 11 determines whether the grip strength of the user acquired by the pressure gauge 131 is equal to or more than the threshold set in step S3 (step S6). The step S6 compares the user's grip strength (threshold) measured by the second kinesiology test (step S3) prior to changing the in-vehicle environment with the user's grip strength measured by the third kinesiology test (step S5) after changing the in-vehicle environment. This determines whether a change in the in-vehicle environment has had a positive or negative effect on the user's body (here, grip).

For example, if a change in the in-vehicle environment positively affects the user's body, the user's grip strength measured by the third kinesiology test (step S5) is greater than or equal to the user's grip strength (threshold) measured by the second kinesiology test (step S3). On the other hand, if the change of the in-vehicle environment negatively affects the user's body, the user's grip strength measured by the second kinesiology test (step S5) will be less than the user's grip strength (threshold) measured by the second kinesiology test (step S3).

In step S6, when it is determined that the grip strength of the user is equal to or more than the threshold value (Yes in step S6), the control unit 11 completes this process. On the other hand, in step S6, when it is determined that the grip strength of the user is less than the threshold value (No in step S6), the control unit 11 returns to step S4 and controls the interface 14 to change the vehicle into another vehicle inside environment. Then, the process is performed after step S5. That is, in the third kinesiology test, when the reaction of the user's body to the in-vehicle environment (grip strength in this case) is not good, until the response of the user's body becomes good, the in-vehicle environment (for example, air conditioner air volume, etc.) is changed little by little without informing the user. At that time, for example, in step S4, when the grip strength is lowered by increasing the air conditioner air volume (when it becomes less than the threshold value), by weakening the air conditioner air volume on the contrary to change the in-vehicle environment.

Here, in step S2, when it is determined that the consciousness level is less than 200 (No in step S2), the control unit 11 transmits the attribute information of the user to the center server 2 (step S7). Subsequently, the center server 2 acquires the in-vehicle environmental information of another user whose attribute information is similar to that of the user from the database 3 (step S8) and transmits the information to the vehicles 1. Subsequently, the control section 11 changes the in-vehicle environment on the basis of the in-vehicle environment information acquired from the center server 2 and returns to the process of step S1.

### First Kinesiology Test

Details of the first kinesiology test will be described with reference to FIG. 3.

First, the control section 11 utters “grasp the steering” to the user through the speaker 142 (step S11). Subsequently, when the user grasps the steering (step S12), the control unit 11 measures the grip strength of the user by the pressure gauge 131 (step S13).

Subsequently, the control unit 11 utters “measures under the best intention” to the user through the speaker 142 (step S14). Subsequently, the control unit 11 utters “the consciousness level is less than 200” through the speaker 142 (step S15). Subsequently, the control unit 11 utters “the consciousness level is 200 or more” through the speaker 142 (step S16).

Subsequently, the grip strength measured after utterance of step S15 (hereinafter referred to as “grip strength of A”) is compared with the grip strength measured after utterance of step S16 (hereinafter referred to as “grip strength of B”) to determine whether the grip strength of B is greater than that of A (step S17).

In step S17, when it is determined that the grip strength of B is larger than the grip strength of A (Yes in step S17), the control unit 11 determines that the consciousness level of the user is 200 or more (step S18) and completes the present process. On the other hand, in step S17, when it is determined that the grip strength of B is equal to or less than the grip strength of A (No in step S17), the control unit 11 determines that the consciousness level of the user is less than 200 (step S19) and completes the present process. Thus, in the first kinesiology test, it is determined whether the user's consciousness level is 200 or more by uttering a predetermined phrase and measuring the grip strength.

### Second Kinesiology Test

Details of the second kinesiology test will be described with reference to FIG. 4.

First, the control section 11 utters “grasp the steering” to the user through the speaker 142 (step S21). Subsequently, when the user grasps the steering (step S22), the control unit 11 measures the grip strength of the user by the pressure gauge 131 (step S23). Subsequently, the control unit 11 sets the value of the grip strength measured in step S23 as the threshold value (step S24), and completes the present process. Thus, in the second kinesiology test, the grip strength of the user prior to changing the in-vehicle environment (step S4) is measured, and the grip strength is set as the threshold.

### Third Kinesiology Test

Details of the third kinesiology test will be described with reference to FIG. 5.

First, the control section 11 utters “grasp the steering” to the user through the speaker 142 (step S31). Subsequently, when the user grasps the steering (step S32), the control unit 11 measures the grip strength of the user by the pressure gauge 131 (step S33), and completes the present process. Thus, in the third kinesiology test, the user's grip strength is measured after the change of the in-vehicle environment (step S4) is made.

Thus, in the vehicle control method according to the embodiment, first, it is determined whether the user's consciousness level is 200 or more by the first kinesiology test (steps S1 and S2), and only when the consciousness level is 200 or more, the subsequent second and third kinesiology tests are performed.

The state of the “consciousness level is 200 or more” indicates a state in which the user's consciousness level is a good state, and the user's body (muscles) responds positively to, for example, a change of an in-vehicle environment (step S4). Thus, if the user's body is at a level of consciousness that responds positively, the second and third kinesiology tests can be performed with high accuracy, and the reliability of the result is also increased. On the other hand, if the user's consciousness level is less than 200, for example, the reaction of the user's body (muscles) to a change in the in-vehicle environment (step S4) becomes negative, which also reduces the accuracy of the second and third kinesiology tests and the reliability of the outcomes.

In the vehicle controller according to the embodiment described above, it is possible to provide an in-vehicle environment according to the desire of the user.

Here, in the vehicle controller according to the embodiment, the case has been described in which the user is a driver of the vehicle 1 as an example. However, the user may be an occupant other than the driver of the vehicle 1. The user may be multiple persons.

In addition, in the first kinesiology test described with reference to FIG. 3, although two phrases were uttered (see steps S15 and S16). However, three or more phrases may be uttered.

In addition, as the biological data of the user used in the first, second, and third kinesiology tests, other than the biological data obtained by the stimulus of five senses, for example, the following data having a correlation with the muscle force may be used.
(1) Body movements (rotates, accelerations), body temperatures, electrocardiograms, pH of perspiration, microscopic RNA, exosomes, simple health checkups, etc.
(2) Number of steps, walking speed, plantar temperature, and plantar force point distribution of the user acquired by biometric shoes
(3) User's normal hand movements, etc. acquired by a bionic glove
(4) Blood flow is read from a change in the color of the user's skin imaged by the camera 132, and the reproduced electrocardiogram data, brain activity data evaluated from a change in the user's facial expression, and the like
(5) User's heart rate, heart rate interval, etc. acquired by the millimeter-wave vital sensor

Further effects and variations can be readily derived by one skilled in the art. Thus, the broader aspects of the invention are not limited to the specific details and representative embodiments represented and described above. Accordingly, various changes may be made without departing from the spirit or scope of the overall inventive concept defined by the appended claims and their equivalents.

According to the present disclosure, it is possible to provide a vehicle controller that can provide an in-vehicle environment according to a user's desire.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A vehicle controller comprising a processor configured to
acquire biological data of a user of a vehicle from a sensor provided in the vehicle;
determine a consciousness level of the user based on the biological by conducting a kinesiology test; and
control an interface mounted on the vehicle in accordance with the determined consciousness level to change an in-vehicle environment.

2. The vehicle controller according to claim 1, wherein
the determining the conscious level includes
outputting a predetermined voice during measurement of the biological data by the sensor, and
determining the consciousness level from the biological data measured after the output of the predetermined voice.

3. The vehicle controller according to claim 2, wherein
the determining the conscious level includes
outputting a first voice that satisfies a predetermined consciousness level, and a second voice that does not satisfy the predetermined consciousness level during the measurement of the biological data by the sensor,
comparing a value of a first biological data measured after the output of the first voice with a value of a second biological data measured after the output of the second voice, and
determining that the conscious level corresponds to the predetermined consciousness level when the value of a first biological data is greater than the value of the second biological data.

4. The vehicle controller according to claim 3, wherein
the changing the in-vehicle environment includes
changing the in-vehicle environment by controlling the interface,
measuring the biological data after the change of the in-vehicle environment,
maintaining the changed in-vehicle environment when the value of the measured biological data is greater than or equal to a predetermined threshold value, and
repeating, when the value of the measured biological data is less than a predetermined threshold value, changing to another in-vehicle environment and measuring the biological data until when the value of the measured biological data is greater than or equal to the predetermined threshold value.

5. The vehicle controller according to claim 1, wherein
the determining the conscious level includes
outputting a first voice that satisfies a predetermined consciousness level, and a second voice that does not satisfy the predetermined consciousness level during the measurement of the biological data by the sensor, and
comparing a value of a first biological data measured after the output of the first voice with a value of a second biological data measured after the output of the second voice, and
the changing the in-vehicle environment includes
changing to in-vehicle environment which was applied to another user having a similar attribute to the user by controlling the interface when the value of the first biological data is less than or equal to the value of the second biological data.

6. The vehicle controller according to claim 1, wherein
the changing the in-vehicle environment includes
maintaining the changed in-vehicle environment when the changed in-vehicle environment is accepted by the user.
